(19) United States
(12) Patent Application Publication
(10) Pub. No.: US 2007/0191875 A1
(43) Pub. Date: Aug. 16, 2007

(54) APPARATUS TO FACILITATE PENETRATION INTO A BODY CAVITY OR LUMEN FORMED BY BODY TISSUE, AND METHOD FOR USING SAME

(76) Inventor: Om Prakash Rehil, Marion, IN (US)

Correspondence Address: TAYLOR & AUST, P.C., 12029 EAST WASHINGTON STREET, INDIANAPOLIS, IN, 46229, US

(21) Appl. No.: 11/468,059

(22) Filed: Aug. 29, 2006

Related U.S. Application Data
(60) Provisional application No. 60/766,894, filed on Feb. 16, 2006.

(51) Int. Cl. A61B 17/34

(52) U.S. Cl. 606/185

(57) ABSTRACT

An apparatus to facilitate penetration into a body cavity or lumen formed by body tissue includes a trocar having a shaft, a handle, and a passageway. The shaft has a longitudinal extent with a first end mounted to the handle and a second end having a sharp point. The passageway extends through the shaft and along the longitudinal extent of the shaft from a first opening located near the first end of the shaft to a second opening located near the second end of the shaft.

### CROSS REFERENCE TO RELATED APPLICATIONS

This is a non-provisional application based upon U.S. provisional patent application Ser. No. 60/766,894, entitled, “DECOMPRESSION ENTEROSTOMY TUBE INSERTION KIT”, filed Feb. 16, 2006.

### FIELD OF THE INVENTION

The present invention relates to a surgical device and procedure, and, more particularly, to an apparatus to facilitate penetration into a body cavity or lumen formed by body tissue, and a method for using the apparatus.

### BACKGROUND OF THE INVENTION

A person with a bowel obstruction usually is very sick and comes to an emergency room for admission as a patient to a hospital. Invariably, their abdomen is markedly distended, tender to palpate, and has distended loops of small and large bowel, depending upon the site and level of bowel obstruction. Nasogastric decompression does help to relieve the gastric distention, but is not effective in relieving the small and large bowel distension, which if not treated appropriately leads to further complications of perforation, bowel ischemia, septicemia, and other complications.

Such patients with bowel obstructions frequently are sent to the operating room as an emergency procedure when they are not in the best of their health, especially when they have multiple other medical problems involving multiple organs. These patients usually are very high surgical and anesthesia risks, and an emergency operation adds to their stress, making the situation worse.

### SUMMARY OF THE INVENTION

The present invention provides apparatus and an associated method to facilitate penetration into a body cavity or lumen formed by body tissue, such as for providing drainage of the body cavity, or for providing decompression of an obstructed lumen of a body part, such as a bowel, stomach, or colon, to reduce internal pressure. For example, in the case of a bowel obstruction, such decompression decreases intra-abdominal pressure, and thus, for example, improves circulation to the bowel, relieves ischemia and swelling of the bowel wall, and improves healing.

The invention, in one form thereof, is directed to an apparatus to facilitate penetration into a body cavity or lumen formed by body tissue. The apparatus includes a trocar having a shaft, a handle, and a passageway. The shaft has a longitudinal extent with a first end mounted to the handle and a second end having a sharp point. The passageway extends through the shaft and along the longitudinal extent of the shaft from a first opening located near the first end of the shaft to a second opening located near the second end of the shaft.

The invention, in another form thereof, is directed to a kit for facilitating penetration of a body cavity or lumen formed by body tissue. The kit includes a trocar configured as described above, and a cannula having an elongate tube for receiving the shaft of the trocar.

The invention, in another form thereof, is directed to a method for facilitating drainage of a body cavity or lumen formed by body tissue. The method includes locating a site on a patient for insertion of a probing apparatus including an assembled trocar and cannula; prepping an area on the skin around the site with skin prep solution; anaesthetizing the area around the site; forming an incision in the skin at the site; and inserting the probing apparatus through the incision and into at least one of the body cavity and the lumen, wherein a portion of the contents in the body cavity or the lumen is drained through a passageway formed in the trocar.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1A is a side view of a penetration apparatus configured in accordance with an embodiment of the present invention.

FIG. 1B is a bottom view of the trocar included in the penetration apparatus of FIG. 1A.

FIG. 1C is a sectional view of the trocar included in the penetration apparatus of FIG. 1A taken along line 1C-1C.

FIG. 1D is a sectional view of the trocar included in the penetration apparatus of FIG. 1A taken along line 1D-1D.

FIG. 1E is a sectional view of an alternative embodiment of the trocar of FIG. 1C.

FIG. 2A is a perspective view, in partial section, of the cannula included in the penetration apparatus of FIG. 1A.

FIG. 2B is a bottom view of the cannula included in the penetration apparatus of FIG. 1A.

FIG. 3A is a side section view of the assembled trocar and cannula of FIG. 1A.

FIG. 3B is a bottom view of the assembled trocar and cannula of FIG. 1A.

FIG. 4A is a side view, in partial section, of a catheter used in practicing a method of the present invention.

FIG. 4B is a bottom view of the catheter of FIG. 4A.

FIGS. 5A and 5B is a flowchart of an exemplary method for decompressing a lumen formed by body tissue, in accordance with an embodiment of the present invention.

FIGS. 6-8 are diagrammatic illustrations of implementing the method depicted in the flowchart of FIG. 5 in relation to various body tissues.

Corresponding reference characters indicate corresponding parts throughout the several views. The exemplifications set out herein illustrate embodiments of the invention, and such exemplifications are not to be construed as limiting the scope of the invention in any manner.

### DESCRIPTION OF THE PREFERRED EMBODIMENT

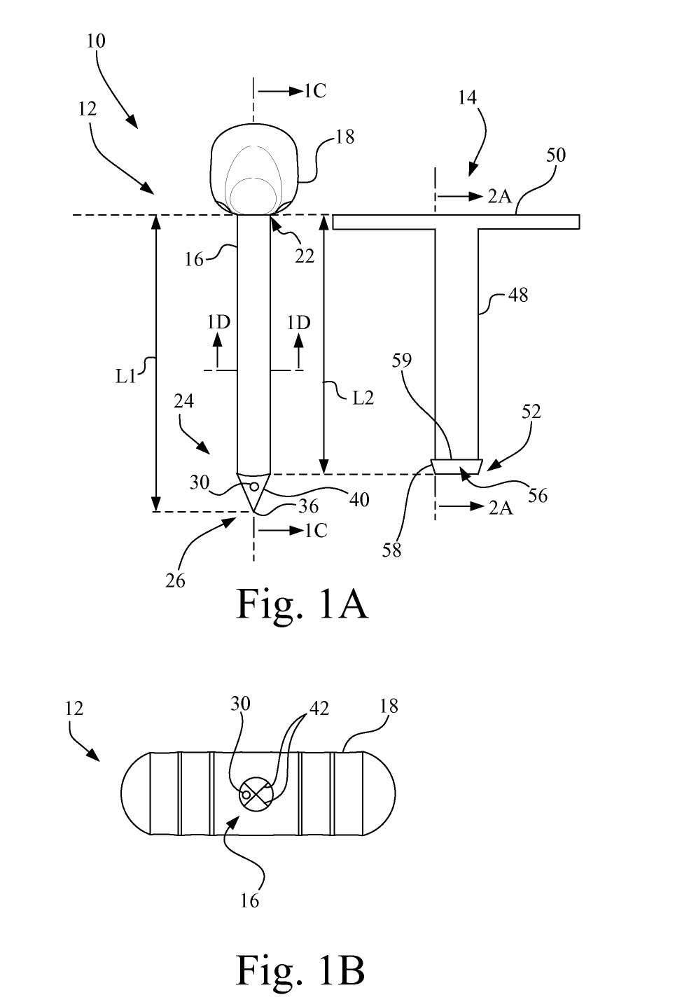

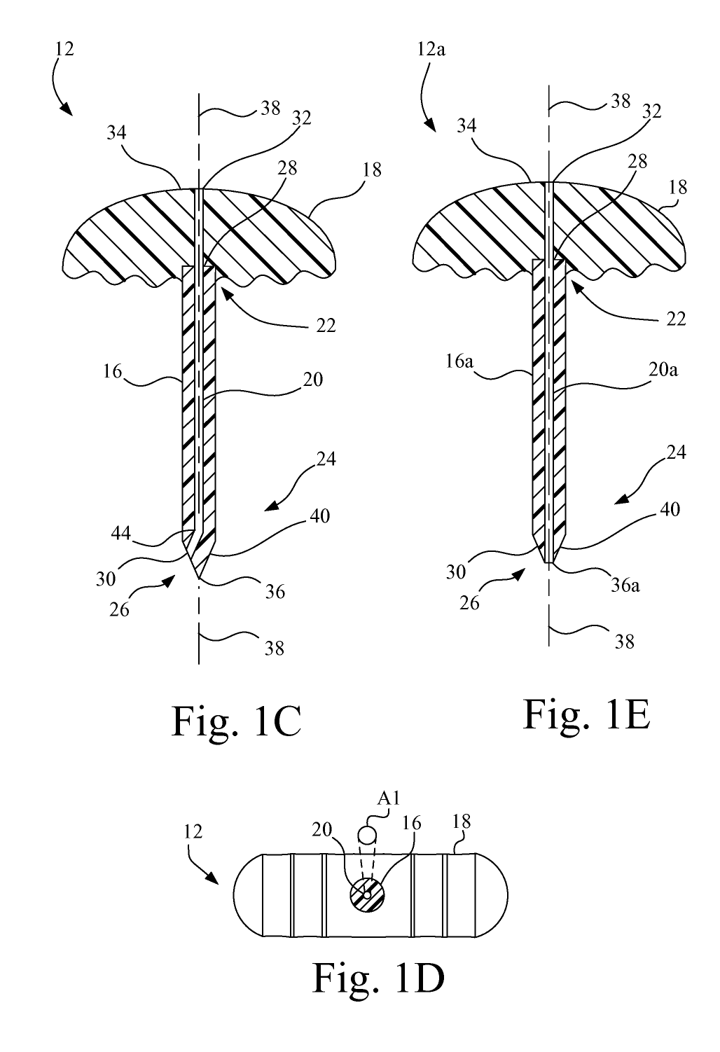

Referring now to the drawings and particularly to FIGS. 1A-3B, there is shown a penetration apparatus 10 configured in accordance with an embodiment of the present invention to facilitate penetration into a body cavity or lumen formed by body tissue. Examples of a body cavity include a chest cavity and an abdominal cavity. Examples of a lumen formed by body tissue include a bowel, stomach, or colon. Penetration apparatus 10 includes a trocar 12, and may further include a cannula 14. Penetration apparatus 10 may be formed from biocompatible materials, such as a plastic.

Trocar 12 has a shaft 16, a handle 18 and a passageway 20. Shaft 16 has a first end 22 mounted to handle 18 and a second end 24 having a sharp point 26. Shaft 16 has a longitudinal extent away from handle 18 of a length L1. Referring to FIG. 1C, passageway 20 extends through shaft 16, and along the longitudinal extent of shaft 16, from an opening 28 located near first end 22 of shaft 16 to an opening 30 located near second end 24 of shaft 16. In the embodiment shown in FIGS. 1A-1D, for example, passageway 20 extends through a portion of handle 18 to form a drain port 32 at an outer surface 34 of handle 18.

In the present embodiment, as illustrated in FIGS. 1A and 1D, the length L1 of shaft 16 may be, for example, about 16.5 centimeters, and passageway 20 may have a cross-sectional area A1 of about 3.2 square millimeters. Those skilled in the art will recognize, however, that length L1 and the cross-sectional area A1 may be determined based on the intended procedure for which penetration apparatus 10 is to be used.

In the present embodiment, opening 30 of passageway 20 is offset from a tip 36 of sharp point 26 of shaft 16 that is located at a centerline 38 of shaft 16. Second end 24 of shaft 16 has a surface 40 that tapers to form sharp point 26. Surface 40 may define one or more cutting edges 42, which are preferably razor sharp. Opening 30 of passageway 20 is located on surface 40, and is spaced away from tip 36 of sharp point 26 of shaft 16, and in turn is spaced away from centerline 38 of shaft 16. Alternatively, in another embodiment, opening 30 may be located at centerline 38 of shaft 16, if desired.

In the present embodiment, a centerline of passageway 20 is substantially coincident with centerline 38 of shaft 16. However, it is contemplated that in an alternative embodiment, the centerline of passageway 20 and centerline 38 of shaft 16 may be offset, if desired. Also, in the present embodiment, passageway 20 may include a slight bend 44, so as to accommodate the location of opening 30 on shaft 16.

FIG. 1E is an alternative embodiment of a trocar 12a, similar in design to trocar 12 of FIG. 1C, except for having a straight path passageway 20a positioned, for example, along centerline 38 of shaft 16a forming a sharp tip 36a.

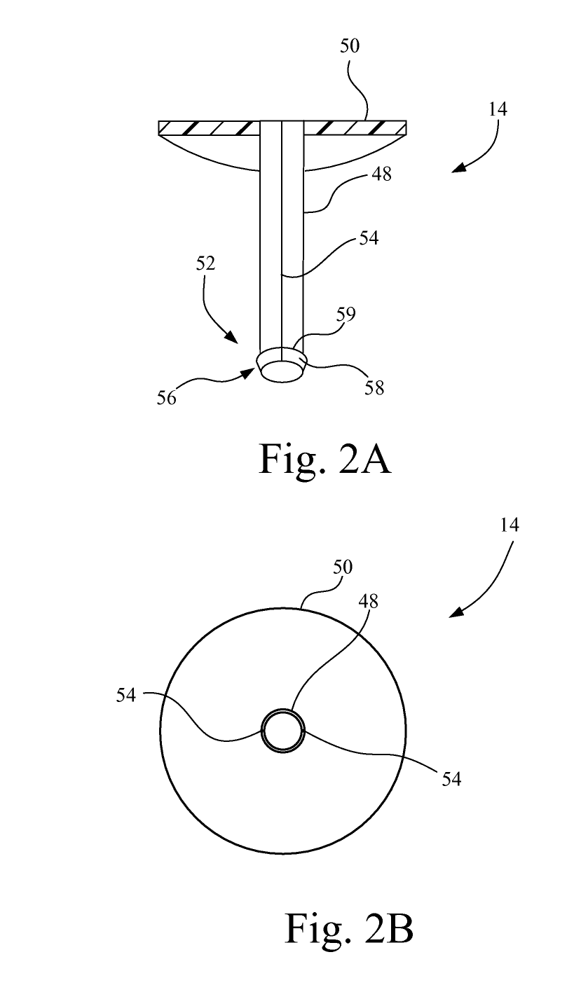

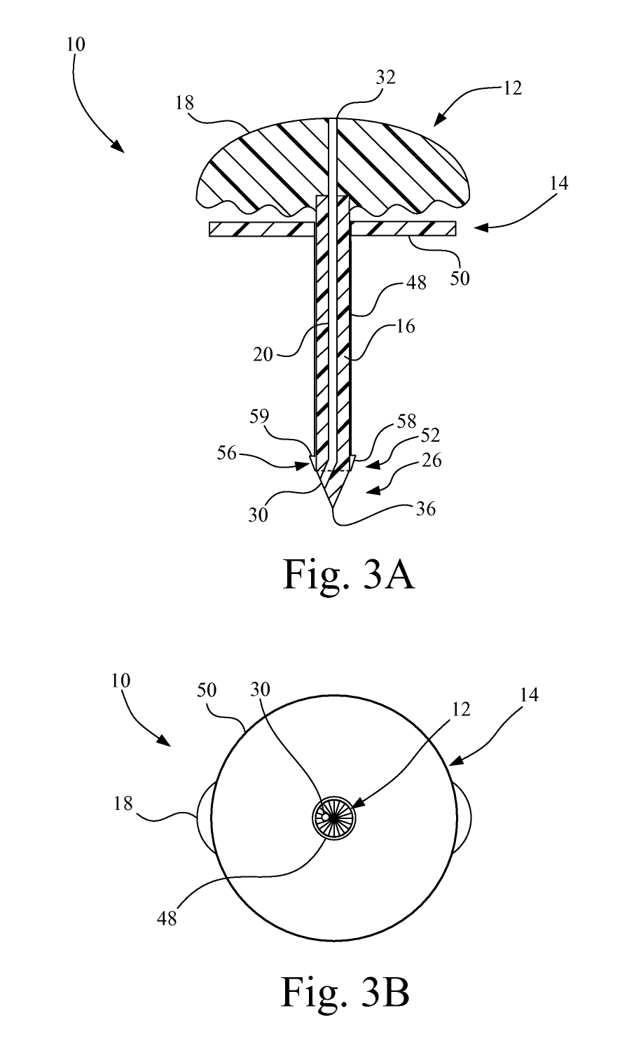

Referring to FIGS. 1A and 2A-3B, cannula 14 is provided to be installed as a sheath over trocar 12. Cannula 14 has an elongate tube 48 for receiving shaft 16 of trocar 12, and an annular member 50 extending radially outwardly from elongate tube 48. Elongate tube 48 has a length L2, selected such that the length L1 of trocar 12 exceeds length L2 of elongate tube 48. Length L2 may be, for example, about 15 centimeters. Accordingly, as illustrated in FIGS. 3A and 3B, when shaft 16 of trocar 12 is completely inserted into elongate tube 48 of cannula 14, the sharp point 26 and opening 30 of trocar 12 both project from a distal end 52 of elongate tube 48 so as to be exposed. Cannula 14 may further have at least two axially extending grooves 54, as illustrated in FIGS. 2A and 2B, defining a split-line for separating cannula 14 into two parts to ease removal of cannula 14 during a procedure.

In the present embodiment, near distal end 52 of cannula 14 there is formed an annular beveled member 56 extending radially outward from elongate tube 48. Annular beveled member 56 has a tapered leading surface 58 to aid in the insertion of cannula 14 through an opening formed in body tissue, e.g., a bowel, and has a blunt trailing surface 59 to resist unintended removal of cannula 14 from the opening in the body tissue, e.g., bowel, in which cannula 14 was inserted.

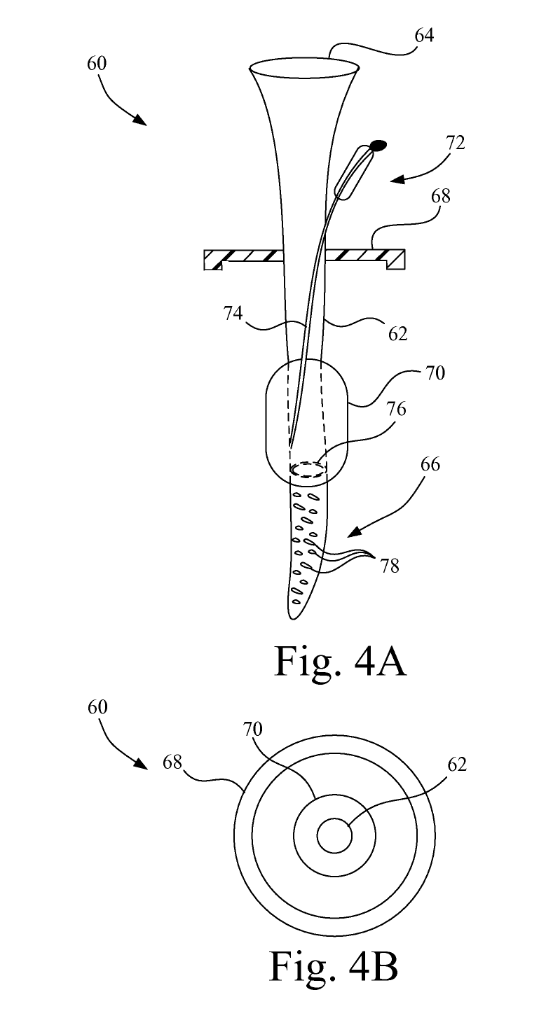

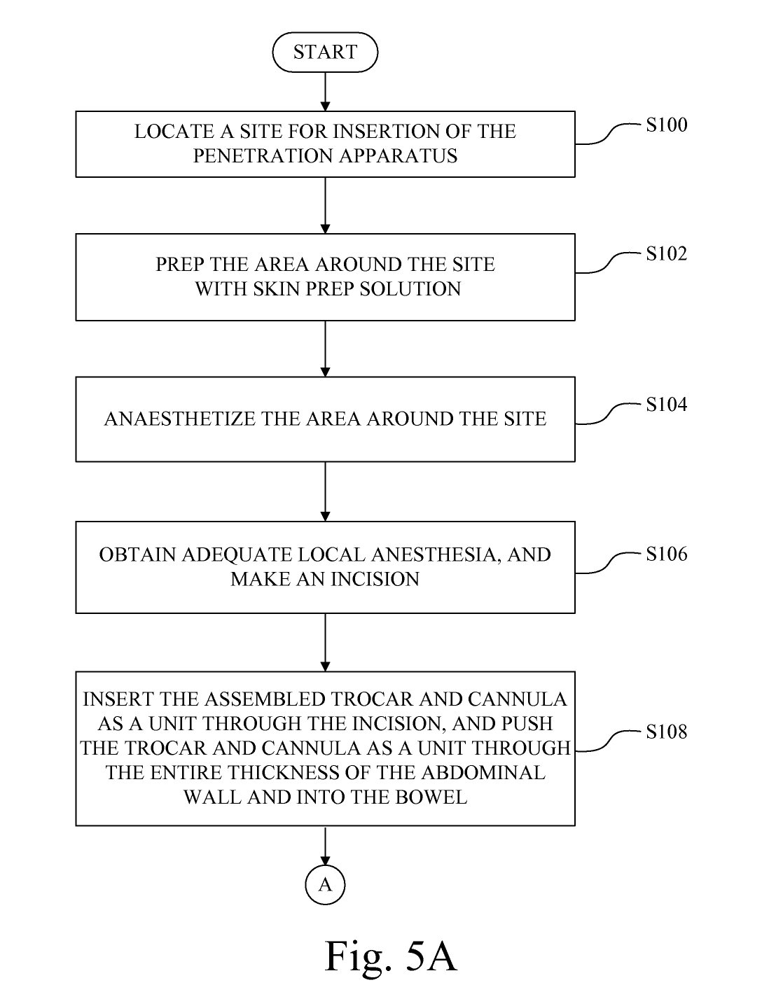

FIGS. 4A and 4B depict an exemplary balloon catheter 60 that may be used in association with the present invention. Balloon catheter 60 may be formed from biocompatible materials, such as a plastic. Balloon catheter 60 includes a tubular shaft 62 having a drainage port 64 and a flexible inlet tip end portion 66. A bolster 68 is positioned in a snug sliding fit over tubular shaft 62, and an inflatable balloon 70 is positioned over and is fixedly attached to tubular shaft 62, with balloon 70 being spaced away from bolster 68 along the length of tubular shaft 62. An inflation valve 72 is attached to balloon 70 via an inflation tube 74.

Balloon catheter 60 forms a central large bore passage 76 for facilitating drainage, such as drainage of a body cavity or human lumen, e.g., to aid in decompression of a bowel, or for instillation of nutrition into the digestive track. Tip end portion 66 is flexible, and is designed to prevent clogging of the central bore passage 76 by having a plurality of inlet apertures 78. The inflatable balloon 70 and bolster 68 cooperate to hold balloon catheter 60 in place, as well as to prevent leakage around tubular shaft 62 of balloon catheter 60. For example, bolster 68 acts as a holding guard to secure balloon catheter 60 in place once bolster 68 is advanced over tubular shaft 62 of balloon catheter 60 to be snug with the outer layer of the body tissue, and also prevents balloon catheter 60 from advancing into the human lumen, e.g., bowel, or body cavity.

In accordance with the present invention, a kit for facilitating penetration of a body cavity or lumen formed by body tissue may include, for example, trocar 12 and cannula 14. The kit may further include, for example, an assortment of supplies for use with trocar 12 and cannula 14, such as sterile drapes and prepping solution. A further addition to the kit may be, for example, balloon catheter 60.

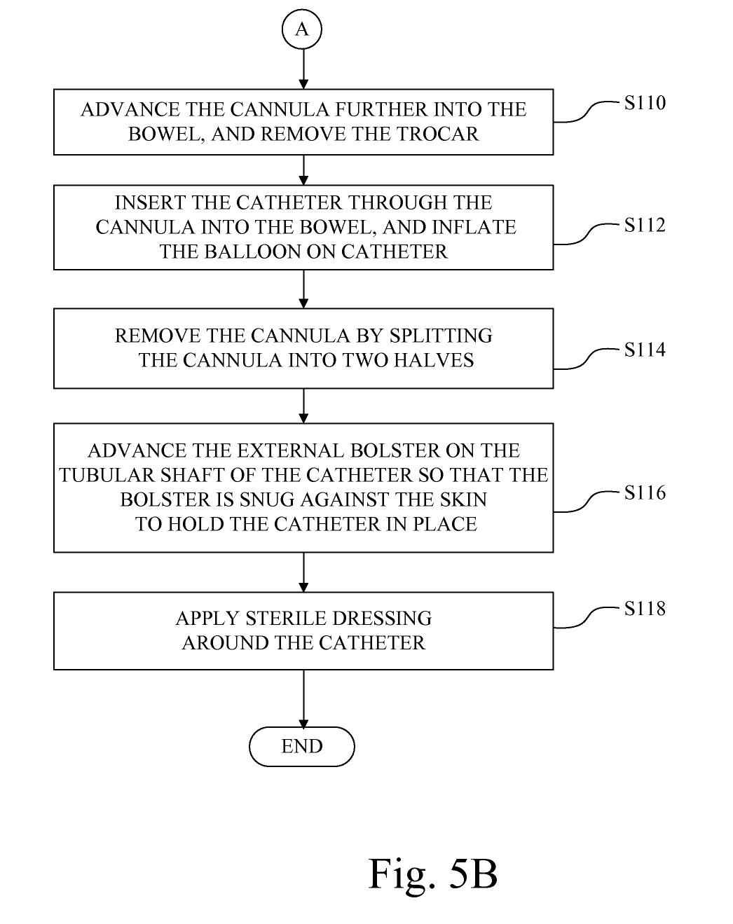

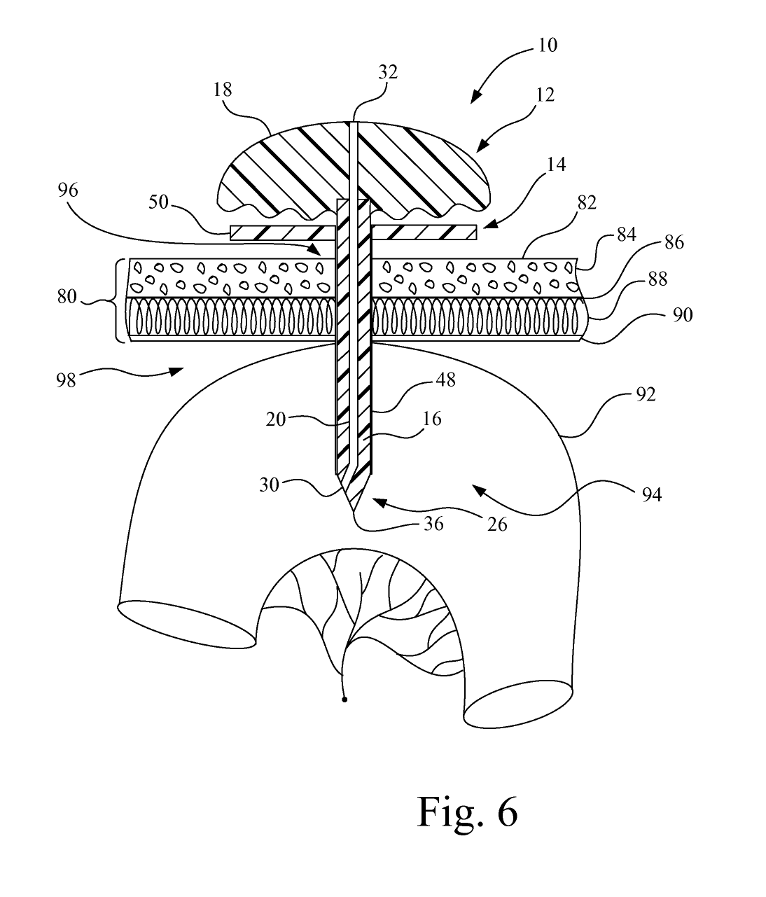

An exemplary method for decompressing a lumen formed by body tissue, in accordance with an embodiment of the present invention, will now be described in relation to the flowchart of FIGS. 5A and 5B, and the illustrations of FIGS. 6-8. Those skilled in the art will recognize that this method may be easily adapted to provide drainage of a body cavity, e.g., by facilitating insertion of a chest tube.

In FIGS. 6, 7, and 8, there is illustrated various body tissues, including an abdominal wall 80 including skin 82, fat 84, fascia 86, muscle 88, and parietal peritoneum 90. Also shown in FIGS. 6-8 is a section of a bowel 92 having a bowel lumen 94.

At step S100, a site 96 is located for insertion of penetration apparatus 10. Usually patients with a bowel obstruction, for example, have a distended abdomen which is packed with distended loops of small and large bowel, depending upon the level of bowel obstruction. The site of insertion of the penetration apparatus 10 can be easily picked based on clinical exam, X-ray findings, Ultrasound guidance, or CAT scan guidance. Most of the distended loops of bowel are in close proximity to the abdominal wall and the area to be decompressed may be selected, most commonly in the right abdomen.

At step S102, the area around site 96 is prepped, e.g., at abdominal wall 80, or the area to be drained or decompressed, with skin prep solution.

At step S104, the area around site 96 is anaesthetized, for example, by injecting 1% xylocain with 1:100,000, with Epinephrine being preferred. However, any local anesthetic can be used. Thus, the process locally anaesthetizes the entire thickness of abdominal wall 80, including all the layers of the abdomen, by injecting the anesthetic in all the layers.

At step S106, after obtaining adequate local anesthesia, an incision, e.g., one and a half centimeters in length, is made in the skin 82.

At step S108, the assembled trocar 12 and cannula 14 are inserted as a unit (e.g., as penetration apparatus 10) through the incision into abdominal wall 80, and with a gentle steady pressure applied to handle 18 of trocar 12, trocar 12 and cannula 14 are pushed through the entire thickness of abdominal wall 80 till the resistance is not encountered any more. The razor sharp tip 36 of trocar 12 cuts through the tissue during penetration, and handle 18 helps to stabilize trocar 12 during penetration. Handle 18 on trocar 12 is designed to allow a good grip and facilitate application of steady pressure during the thrusting of trocar 12 through the tissues.

Trocar 12 and cannula 14 will penetrate through the entire thickness of abdominal wall 80 into the distended bowel 92, and a portion of the intestinal contents in bowel 92 will drain by flowing into opening 30, through passageway 20, and will be expelled out of drain port 32, thereby suggesting that trocar 12 and cannula 14 are into the bowel lumen 94. If desired, or necessary, trocar 12 may be installed over a guide wire threaded through passageway 20.

At step S110, cannula 14 is then advanced further into bowel 92 gently, and trocar 12 is removed. At this juncture the intestinal contents in bowel 92 will come out through the cannula 14 freely.

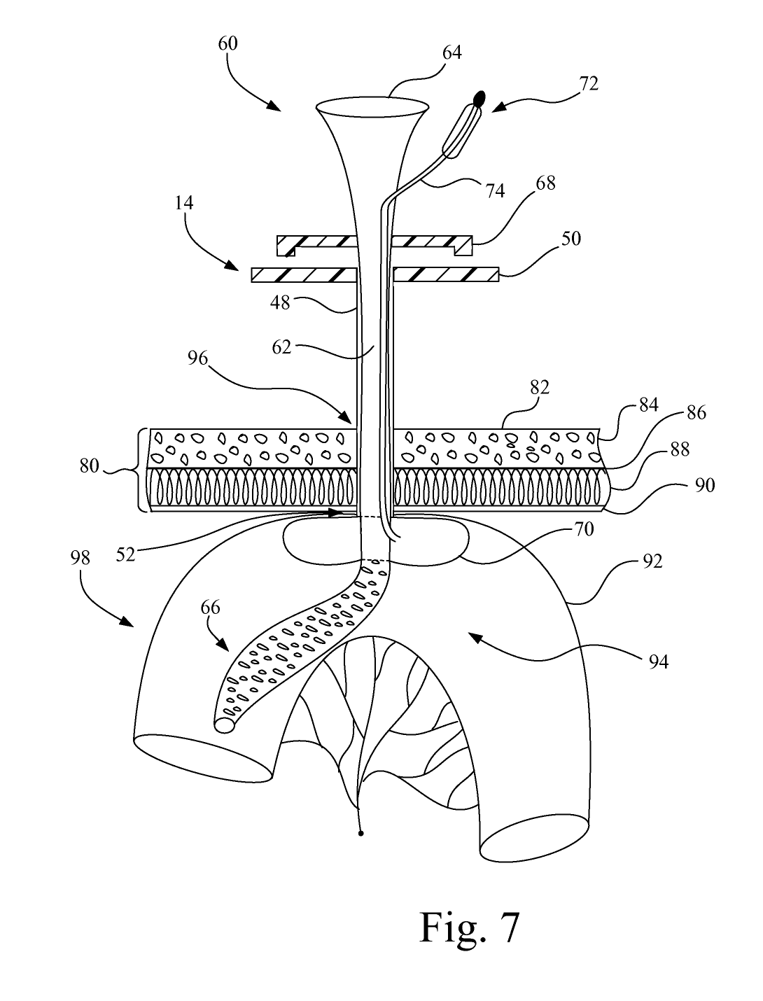

At step S112, referring to FIG. 7, catheter 60 is inserted through cannula 14 into bowel 92, and balloon 70 on catheter 60 is then gently inflated. Caution should be taken to insure that catheter 60 is advanced into bowel lumen 94 beyond distal end 52 of elongate tube 48 of cannula 14 before balloon 70 is insufflated. Catheter 60 is then gently pulled outwardly away from abdominal wall 80 till balloon 70 is pulling the wall of bowel 92 against the parietal peritoneum 90, thus preventing any leakage of the intestinal contents in bowel 92 into the peritoneal cavity 98, and thus avoiding the contamination of the free peritoneal cavity 98. The capacity of balloon 70 is fixed so it does not mechanically obstruct the lumen, especially, for example, when catheter 60 is used for feeding rather than for decompression of bowel 92.

At step S114, cannula 14 is removed by splitting cannula 14 into two halves along groove 54 (see FIGS. 2A and 2B).

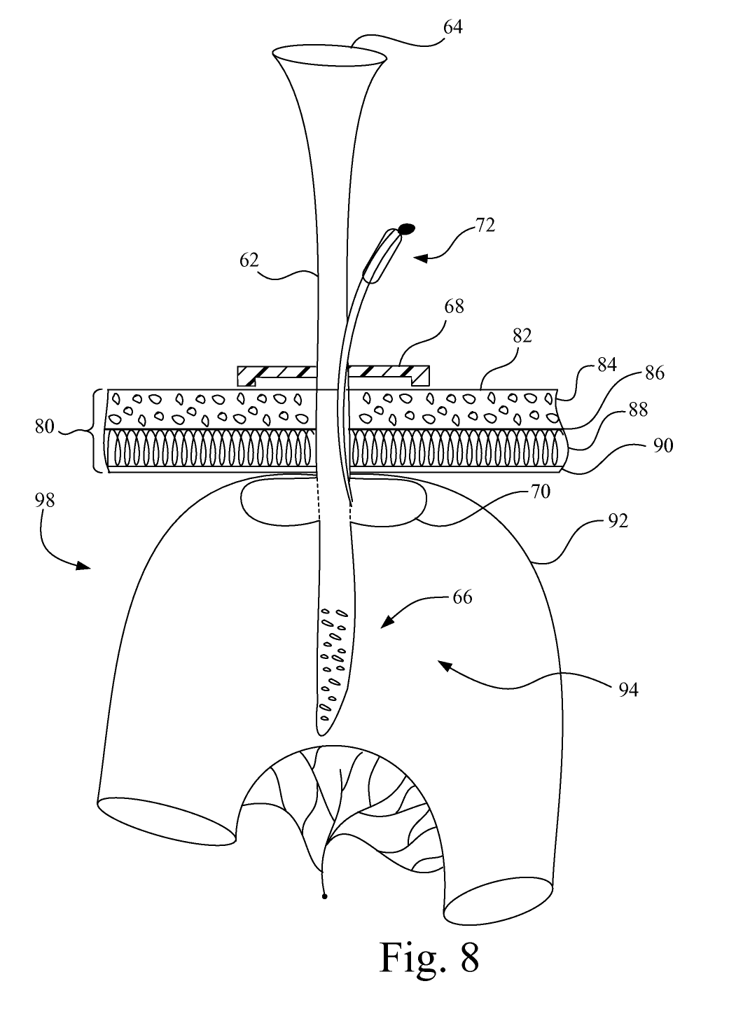

At step S116, referring to FIG. 8. the external bolster 68 is gently advanced on tubular shaft 62 of the drainage catheter 60 so that bolster 68 is snug against the skin 82 to hold the catheter 60 in place. Silk suture is tied around bolster 68 to prevent any advancement of drainage catheter 60 into bowel 92.

At step S118, sterile dressing is applied around catheter 60, and catheter 60 is ready to decompress bowel 92 once attached to any kind of suction device. Alternatively, catheter 60 may be left open, e.g., unattached to a suction device, for gravity drainage. Catheter 60 may be irrigated easily, is desired, to keep catheter 60 patent and proper functioning.

Advantages of the present invention are numerous, with some being listed below by way of example, and not limitation. For example, the decompression procedure of the present invention is relatively simple to perform and may be accomplished with relative ease, thereby lessening the need for a major procedure as an emergency surgery in a sick high risk surgical patient. The patient may thus be stabilized for definitive care and treatment after correcting the nutritional status, electrolyte imbalance and prepping the bowel in case it is needed. Also, decompressing the bowel in an obstructed patient will improve the respiratory status by reducing the pressure on the diaphragm, thus improving ventilation.

Also, a decompressed bowel is easier to manipulate during the surgical procedure and will reduce operation time by facilitating fast exploration and quick decision to get to the pathological problem and take care of it appropriately. The decompression of the distended bowel occurs proximal to the obstruction without major surgical intervention, thus giving an opportunity to the surgeon to plan the elective procedure. The procedure may be done at the patient's bed site under local anesthesia, and will help to improve the general condition of the patient by improving nutrition, electrolyte imbalance and help to stabilize the patient before subjecting the patient to a major operation.

Examples for use of the present invention include, without limitation: with small bowel obstruction secondary to adhesions, volvulus, internal hernias, tumors, inflammatory bowel disease; with prolonged paralytic ileus, prolonged non resolving post operative ileus warranting decompression; with colon obstruction secondary to obstruction, volvulus, tumors inflammatory bowel disease, obstipation, neurological causes causing colon distention; to drain localized intraabdominal abscesses without major intervention; facilitating drainage of intrabbdominal cysts or ascites; facilitating drainage of localized empyema or hemothorax; open feeding gastrostomy or jejunostomy; and in an insertion of a chest tube.

While this invention has been described with respect to embodiments of the invention, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A apparatus to facilitate penetration into a body cavity or lumen formed by body tissue, comprising a trocar having a shaft, a handle, and a passageway, said shaft having a longitudinal extent with a first end mounted to said handle and a second end having a sharp point, said passageway extending through said shaft and along said longitudinal extent of said shaft from a first opening located near said first end of said shaft to a second opening located near said second end of said shaft.

2. The apparatus of claim 1, wherein said passageway extends through a portion of said handle to an outer surface of said handle.

3. The apparatus of claim 1, wherein said second opening of said passageway is offset from a tip of said sharp point of said shaft.

4. The apparatus of claim 1, wherein said second end of said shaft has a surface that tapers to form said sharp point, said second opening of said passageway being located on said surface and spaced away from a tip of said sharp point of said shaft.

5. The apparatus of claim 1, further comprising a cannula having an elongate tube for receiving said shaft of said trocar, said shaft of said trocar having a first length and said elongate tube of said cannula having a second length, said first length exceeding said second length, such that when said shaft of said trocar is completely inserted into said elongate tube, said sharp point and said second opening of said trocar project from a distal end of said elongate tube so as to be exposed.

6. The apparatus of claim 5, said cannula having an annular beveled member extending radially outwardly from said elongate tube.

7. The apparatus of claim 5, said cannula having at least two axially extending grooves defining a split-line for separating said cannula into two parts.

8. A kit for facilitating penetration of a body cavity or lumen formed by body tissue, comprising:
a trocar having a shaft, a handle, and a passageway, said shaft having a longitudinal extent with a first end mounted to said handle and a second end having a sharp point, said passageway extending through said shaft and along said longitudinal extent of said shaft from a first opening located near said first end of said shaft to a second opening located near said second end of said shaft; and
a cannula having an elongate tube for receiving said shaft of said trocar.

9. The kit of claim 8, further comprising an assortment of supplies for use with said trocar and said cannula, including sterile drapes and prepping solution.

10. The kit of claim 8, further comprising a balloon catheter sized for insertion through said elongate tube.

11. A method for facilitating drainage of a body cavity or lumen formed by body tissue, comprising:
locating a site on a patient for insertion of a probing apparatus including an assembled trocar and cannula;
prepping an area on the skin around said site with skin prep solution;
anaesthetizing said area around said site;
forming an incision in the skin at said site; and
inserting said probing apparatus through said incision and into at least one of said body cavity and said lumen, wherein a portion of the contents in said body cavity or said lumen is drained through a passageway formed in said trocar.

12. The method of claim 11, further comprising:
removing said trocar from said cannula; and
inserting a catheter in said cannula where said trocar was previously located.

13. The method of claim 12, wherein said catheter is a balloon catheter, said method further comprising:
inflating a balloon of said balloon catheter;
pulling said catheter outwardly away from said patient till said balloon is pulling a wall of one of said body cavity or said lumen to reduce the possibility of leakage of said contents internal to said patient;
splitting said cannula to aid in removing said cannula; and
advancing an external bolster on a tubular shaft of said catheter until said bolster is snug against the skin of said patient.

14. The method of claim 13, further comprising applying a vacuum to said balloon catheter to aid in draining said contents.

15. The method of claim 13, further comprising draining said contents with said balloon catheter by gravitational force.

16. The method of claim 13, further comprising applying a sterile dressing around said balloon catheter.
